(19) United States
(12) Patent Application Publication
(10) Pub. No.: US 2019/0117810 A1
(43) Pub. Date: Apr. 25, 2019

(54) READERS FOR PROCESS MONITORING SYSTEMS AND METHODS OF USE

(71) Applicant: 3M INNOVATIVE PROPERTIES COMPANY, SAINT PAUL, MN (US)

(72) Inventors: PETER D. LUDOWISE, COTTAGE GROVE, MN (US); ANTHONY J. JENNEN, VADNAIS HEIGHTS, MN (US); BARRY W. ROBOLE, WOODVILLE, WI (US); PHILIP G. DION, BLAINE, MN (US); LEROY J. LONGWORTH, WOODBURY, MN (US); JAY R. GOETZ, DEEPHAVEN, MN (US)

(73) Assignee: 3M INNOVATIVE PROPERTIES COMPANY, SAINT PAUL, MN (US)

(21) Appl. No.: 16/094,511

(22) PCT Filed: Apr. 19, 2017
(86) PCT No.: PCT/US17/28236

§ 371 Date: Oct. 18, 2018

Related U.S. Application Data
(60) Provisional application No. 62/326,323, filed on Apr. 22, 2016.

(51) Int. Cl. A61L 2/24; G01N 21/27; G01N 21/84; G01N 21/94

(52) U.S. Cl. CPC A61L 2/24; G01N 21/94; G01N 21/8483; G01N 21/27

(57) ABSTRACT

Process monitoring systems include a reader having a read sensor, an optional reference sensor, and a light source. The reader optionally includes an optical block defining an interior volume and an aperture aligned with the read sensor to allow diffuse light to be received by the read sensor. One or more percent reflectance values may be calculated in response to a dark measurement, a correction function, and normalization. The one or more percent reflectance values may be compared to a threshold to determine whether a disinfection cycle was effective. The reader may indicate the determination to a user and provide the determination to a tracking system for diagnostics, inventory management, or compliance monitoring.

### FIELD

The present disclosure generally relates to readers for use with process monitoring systems, and particularly, for use with process monitoring systems configured to monitor endoscope reprocessing systems, as well as related methods of use.

### BACKGROUND

Endoscopy procedures play a beneficial role in the prevention, diagnosis and treatment of disease. Endoscopy procedures are performed using complex, reusable, flexible instruments that, when inserted into the body, may become heavily contaminated with patient biomaterial and microorganisms, including potential pathogens. Careful reprocessing of flexible endoscopes between patients is critical to reducing the risk of cross-contamination and the possible transmission of pathogens.

Flexible endoscopes are rated as semi-critical according to the Spaulding classification for medical devices and therefore it is required that these devices be decontaminated by high-level disinfection. Thus, it is recommended that both endoscopes and reusable accessories be frequently visually inspected in the course of their use and reprocessing, including before, during and after use, as well as after cleaning and before high-level disinfection. However, a visually based method of verification has severe limitations when applied to flexible endoscopes because the complex, narrow lumens in these devices cannot be directly visually inspected.

Automated endoscope reprocessors (AERs) are used to clean and disinfect flexible endoscopes to a level that mitigates transmission of pathogenic organisms and disease between patients who are subject to an endoscopic procedure. Typically, the only information available to a user is the parametric information provided by the AER equipment itself which consists primarily of time and temperature information. The AER does not monitor chemical parameters capable of establishing the effectiveness of the disinfection cycle.

Existing procedures to verify the effectiveness of the disinfection cycle with chemical or biological indicators for use with AERs rely on visual inspection that may be susceptible to subjective interpretation.

### SUMMARY

The present disclosure relates to process monitoring systems that indicate whether a process was effective. In particular the disclosure relates to an apparatus, system, and method for indicating whether a change in the color of a process indicator is representative of a successful disinfection cycle in a processing system.

In one illustrative embodiment, a system includes a reader configured to indicate whether an indicator surface of a process indicator has a color representative of an effective disinfection cycle when the process indicator is in a reading position of the reader. The process indicator defines an axis normal to the indicator surface. The reader includes an optical block defining an interior volume and an aperture. The axis extends through the aperture when the process indicator is in the reading position. The reader also includes a light source disposed in the interior volume and configured to illuminate the indicator surface with light. The axis does not extend through the light source when the process indicator is in the reading position. Optionally, the reader includes a reference sensor positioned in the interior volume to receive, at a reference sensor surface, light from the light source that has not passed through the aperture. The reference sensor is configured to provide a reference sensor signal value representing an intensity of light received. The axis does not extend through the reference sensor surface when the process indicator is in the reading position. The reader further includes a read sensor disposed in the interior volume to receive, at a read sensor surface, light from the light source that has reflected off the indicator surface and has passed through the aperture. The read sensor is configured to provide a read sensor signal value representing an intensity of reflected light received. The reader additionally includes a processor operatively coupled to the light source, read sensor, and optional reference sensor. The processor is configured to receive the read sensor signal value and optionally the reference sensor signal value. The processor is also configured to determine whether the process indicator has the color representative of an effective disinfection cycle based at least in part on the read sensor signal value and optionally the reference sensor signal value.

In another illustrative embodiment, a method includes generating a dark read sensor signal value from a read sensor and optionally a dark reference sensor signal value from an optional reference sensor after a process indicator is in a reading position of a reader. The method also includes selectively turning on a light source of the reader at different wavelengths including a first wavelength and a second wavelength. The method further includes generating, for each of the different wavelengths, a corresponding read sensor signal value and optionally a corresponding reference sensor signal value. Each read sensor signal value represents an intensity of light reflected from the process indicator in the reading position and received by the read sensor. Each reference sensor signal value represents an intensity of light received by the reference sensor from the light source. The method additionally includes generating percent reflectance values, including a first percent reflectance value for the first wavelength and a second percent reflectance value for the second wavelength. Each percent reflectance value is based at least in part on the corresponding read sensor signal value, the dark reference sensor signal value, the dark read sensor signal value, a corresponding predetermined correction function, and optionally the corresponding reference sensor value. Also, the method includes normalizing the first percent reflectance value based at least in part on the second percent reflectance value. Further, the method includes determining whether the process indicator exhibits a color representative of an effective disinfection cycle based at least in part on the normalized first percent reflectance value and a corresponding predetermined threshold.

In a further illustrative embodiment, an apparatus includes a light source configured to illuminate a reading area with light at different wavelengths including a first wavelength and a second wavelength. The apparatus also includes a read sensor positioned to receive light from the reading area. The read sensor is configured to provide a read sensor signal value representing an intensity of light received. The read sensor optionally includes a reference sensor positioned to receive light from the light source. The reference sensor is configured to provide a reference sensor signal value representing an intensity of light received at the reference sensor surface. The reader further includes a processor operatively coupled to the light source, read sensor, and optional reference sensor. The processor is configured to selectively turn on the light source at the different wavelengths. The processor is also configured to receive, for each of the different wavelengths, a read sensor signal value and optionally a reference sensor signal value. The processor is additionally configured to determine whether a disinfection cycle was effective based at least in part on the read sensor signal values and optionally the reference sensor signal values.

These and various other features and advantages will be apparent from a reading of the following detailed description.

Additional features and advantages of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure, and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative, and are not intended to limit the scope of the claims in any manner.

### BRIEF DESCRIPTION OF THE DRAWINGS

The disclosure may be more completely understood in consideration of the following detailed description of various embodiments of the disclosure in connection with the accompanying drawings.

FIG. 1 is a schematic perspective view of a reprocessing system with a process monitoring system according to one embodiment of the present disclosure.

FIG. 2 is a schematic perspective view of a reader and a process indicator according to one embodiment of the present disclosure.

FIG. 3 is a schematic bottom view of a printed circuit board of the reader of FIG. 2 according to one embodiment of the present disclosure.

FIGS. 4-5 are schematic, cross-sectional elevation views of the reader of FIG. 2 along line 4-4 according to one embodiment of the present disclosure.

FIG. 6 is a schematic top view of an optical block of the reader of FIG. 2 according to one embodiment of the present disclosure.

FIG. 7 is a schematic representation of a processing monitoring system according to one embodiment of the present disclosure.

FIG. 8 is a schematic representation of the wavelength spectra of a light source of a reader according to one embodiment of the present disclosure.

FIG. 9 is a schematic representation of a process of measuring with a reader according to one embodiment of the present disclosure.

FIG. 10 is a schematic representation of calibrating a reader according to one embodiment of the present disclosure.

FIG. 11 is a schematic representation of a process of calibrating a reader according to one embodiment of the disclosure.

FIGS. 12A-B are schematic representations of normalizing signal values according to one embodiment of the present disclosure. FIG. 12A representing signal values before normalization, and FIG. 12B representing normalized signal values.

FIG. 13 is a schematic representation of a process of using the reader according to one embodiment of the present disclosure.

### DETAILED DESCRIPTION

The present disclosure generally relates to readers for user with process monitoring systems, process monitoring systems including such readers, and related methods.

All scientific and technical terms used herein have meanings commonly used in the art unless otherwise specified. The definitions provided herein are to facilitate understanding of certain terms used frequently herein and are not meant to limit the scope of the present disclosure.

Process monitoring systems of the present disclosure, and elements thereof, can be used to monitor the effectiveness of a variety of processing systems including various cleaning, disinfecting, and/or sterilization processes or systems (e.g., reprocessing systems). For example, in some embodiments, process monitoring systems of the present disclosure can be used to monitor an endoscope reprocessing system. Such an endoscope reprocessing system can include, but is not limited to, an automated endoscope reprocessor (AER), an endoscope cleaning reprocessor (ECR), a liquid chemical sterilization (LCS) system, or the like, or a combination thereof. By way of example only, the process monitoring systems of the present disclosure can be particularly useful for monitoring the effectiveness of a disinfection cycle provided by an AER. As a result, the readers, indicators, and systems of the present disclosure are sometimes described herein with reference to use with an AER. However, it should be understood that the readers, indicators, and systems of the present disclosure can be used in monitoring other endoscope reprocessing systems, as well as other cleaning, disinfecting, and/or sterilization processes or systems.

Process monitoring systems may include tracking systems coupled in operative communication with the reader. Readers may indicate whether a color change represents an effective disinfection cycle to tracking systems. Tracking systems may perform a number of tasks, such as providing diagnostic assistance for ineffective disinfection cycles, managing the inventory of disposable process indicators, maintaining a database of disinfection events, etc.

Reference will now be made to the drawings, which depict one or more aspects described in this disclosure. However, it will be understood that other aspects not depicted in the drawings fall within the scope of this disclosure. The following detailed description, therefore, is not to be taken in a limiting sense.

For example, like numbers used in the figures refer to like components, steps and the like. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

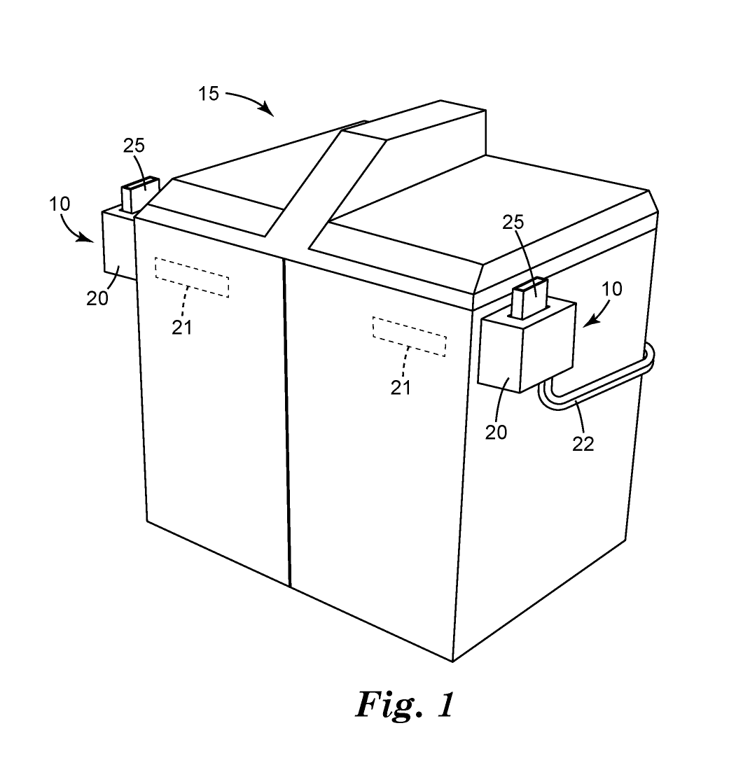

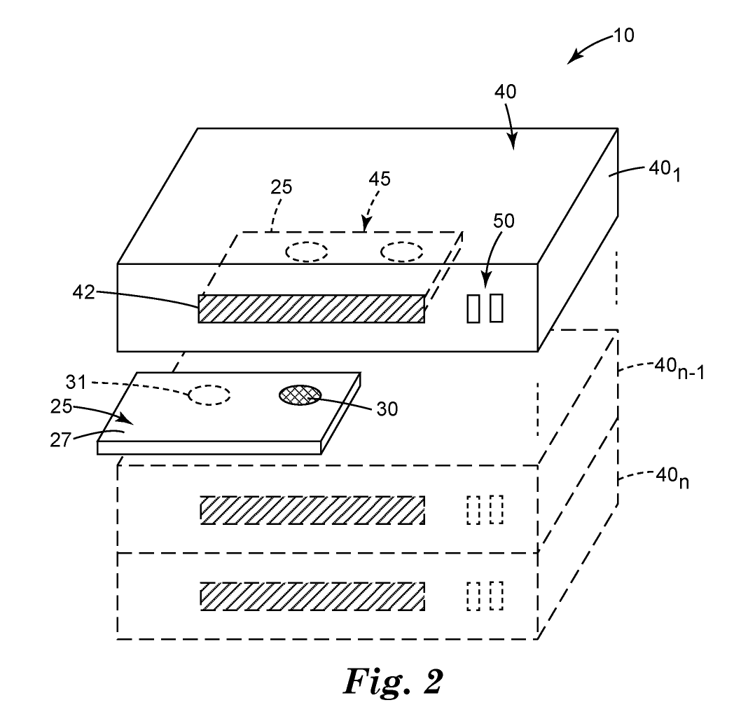

As illustrated in FIG. 1 and FIG. 2, in one illustrative embodiment, a process monitoring system 10 is externally secured to a processing system 15, as shown in FIG. 1. The processing system 15 can be configured to couple to equipment (e.g., an endoscope) and to perform a disinfection cycle for cleaning the equipment. The process monitoring system 10 can include one or more adapters 20 coupled to the processing system 15, and a cartridge 25 can be coupled to each adapter 20. A process indicator 30 can be included in each cartridge 25 for verifying the presence of conditions for effective disinfection, as shown in FIG. 2, and can be used to determine if a disinfection cycle was effective. A reader 40 may be configured to read a process indicator 30 in a reading position 45 of the reader to indicate whether the process indicator 30 represents an effective disinfection cycle, for example, via observable indicators 50.

One or more pieces of equipment may be coupled to the processing system 15, for example, to be subjected to a disinfection cycle, a sterilization cycle, and/or a cleaning cycle. In the illustrated embodiment, the processing system 15 is capable of coupling to two pieces of equipment and performing two disinfection cycles concurrently.

The processing system 15 may include one or more solution reservoirs (not shown), which may be disposed within the processing system 15. The processing system 15 can be configured to fluidly couple equipment with the one or more solution reservoirs. Non-limiting examples of solution reservoirs include a disinfectant solution reservoir and a detergent solution reservoir. In a disinfection cycle, a disinfectant solution may be pumped from a disinfectant solution reservoir and introduced to disinfectable equipment.

Certain disinfection conditions may be desirable to facilitate an effective disinfection cycle. The processing system 15 can be designed to perform a disinfection cycle according to one or more disinfection conditions. However, the processing system 15 may not be able to verify that the desired disinfection conditions were achieved.

In some embodiments, a particular concentration of disinfectant solution may be desired for the disinfection cycle to be effective. However, in various embodiments, the processing system 15 may not be capable of verifying that the equipment is being exposed to a sufficient concentration of disinfectant solution for the disinfection cycle to be effective.

In some embodiments, a disinfectant solution may be heated by the processing system 15 until reaching a desired temperature for the disinfectant solution to be effective. However, in various embodiments, the processing system 15 may not be capable of verifying the temperature that the equipment was exposed to disinfectant solution.

In some embodiments, a disinfectant solution may be introduced to the equipment for a desired exposure time for the disinfectant solution to be effective. However, in various embodiments, the processing system 15 may not be capable of verifying the exposure time that the equipment was exposed to disinfectant solution.

In the illustrated embodiment, the process monitoring system 10 includes two adapters 20 coupled to the processing system 15. Each adapter 20 can include a fluid pathway and be coupled in fluid communication with equipment in the processing system 15. As shown, for example, one or more tubes 22 can fluidly connect the adapter 20 to equipment in the processing system 15. The adapters 20 may be fluidly coupled in any suitable manner, for example, in series or in parallel to the equipment being disinfected in the processing system 15 (e.g., an endoscope).

Each adapter 20 can be coupled in fluid communication with the processing system 15 and the equipment. In some embodiments, an adapter 20 may be coupled to the disinfectant solution reservoir and/or a piece of equipment. In one example, an adapter 20 may be fluidly coupled to the disinfectant solution reservoir of the processing system 15 via a first tube 22 and fluidly coupled to equipment via a second tube 22.

The adapters 20 can be disposed externally to the processing system 15 as shown in the illustrated embodiment. However, the processing system 15 may additionally or alternatively include adapters 21 disposed internally in the processing system 15. Adapters 21 may be similar to adapters 20 except that the adapters 21 are at least partially disposed internally within the processing system 15.

The process monitoring system 10 may be separate or integrated with the processing system 15. In some embodiments, the processing system 15 may be considered to include the process monitoring system 10.

The process indicator 30 may be positioned in fluid communication with the adapter 20. After a process indicator 30 is positioned in fluid communication with the adapter 20, a disinfection cycle may be run on the processing system 15.

In various embodiments, during a disinfection cycle, the process indicator 30 is preferably subjected to the same or similar conditions as the equipment being processed in the processing system 15. The process indicators 30 are preferably configured to indicate whether a disinfection cycle was effective, for example, by being responsive to various disinfection conditions.

The process indicator 30 may be only a portion of the cartridge 25. Each cartridge 25 may include one process indicator 30 or more than one process indicator 30, each of which may be the same or different (e.g., configured to respond to different disinfection conditions).

The process indicator 30 can be a color change-based indicator. The degree of color change may be characterized by the reflectance (or inversely, the absorption) of the process indicator 30 at one or more wavelengths of light. In some embodiments, process indicators 30 are configured to change color when subjected to one or more disinfection conditions. Non-limiting examples of disinfection conditions include exposure time, temperature, disinfectant solution concentration, etc., and combinations of two or more thereof.

In some embodiments, the process indicator 30 can also be configured to exhibit a particular color (e.g., changed color) when subjected to a particular type of disinfectant solution, which may include a high level disinfectant (HLD) for reprocessing, for example. In some embodiments, the process indicator 30 can change color when subjected to ortho-phthaldehyde (OPA). In some embodiments, the process indicator 30 can change color when subjected to glutaraldehyde (GA). In some embodiments, the process indicator 30 can change color when subjected to peracetic acid. In some embodiments, the process indicator 30 can change color when subjected to hypochlorous acid/hypochlorite. In some embodiments, the process indicator 30 can change color when subjected to hydrogen peroxide. In some embodiments, the process indicator 30 can change color when subjected to any combination of two or more high level disinfectants, listed above. For example, the process indicator 30 may change color to a first reflectance profile when subjected to OPA (e.g., white to yellow) and to a second reflectance profile when subjected to GA (e.g., white to orange).

The process indicator 30 may be configured to change color when subjected to more than one disinfection condition. Such an indicator may be described as an integrator. For example, a piece of endoscope equipment may preferably be subjected to OPA at a minimum effective concentration of 0.35% by volume (e.g., which may be a commercially-available high-level disinfectant diluted in water) at a temperature of 25° C. for 10 minutes. The color of the process indicator 30 may not represent a successful disinfection cycle unless all conditions of concentration, temperature, and time are met.

The process indicator 30 may, in one or more embodiments, be a chemical indicator disposed on a thin piece of paper attached to a body 27 of the cartridge 25. The cartridge body 27 may have a depth defining the height of the flow path through the cartridge 25. In some embodiments, the process indicator 30 may be disposed near a top or bottom surface of the cartridge 25 within the flow path.

The cartridges 25 may be removably coupled to the adapters 20. In the illustrated embodiment, each cartridge 25 is removably inserted into an adapter 20. Once a cartridge 25 having a process indicator 30 has been removed from an adapter 20, the process indicator 30 of the cartridge 25 may be positioned in a reading position of the reader. As shown in FIG. 2, in the illustrated embodiment, the reader 40 is designed to receive the cartridge 25 into a slot 42 to place the process indicator 30 in a reading position 45 with respect to the reader 40.

The reader 40 can be configured to read the process indicator 30 in the reading position 45. For example, the reader 40 may determine whether a process indicator 30 has a color representative of an effective disinfection cycle when the process indicator 30 is in a reading position 45 of the reader 40.

Observable indicators 50 may be included with the reader 40. A first observable indicator 50 may be included to inform a user that the disinfection cycle was effective (e.g., a green light may be activated). A second observable indicator 50 may also be included to inform a user that a disinfection cycle was ineffective (e.g., a red light may be activated).

Each process monitoring system 10 may include more than one reader 40 to read two or more different process indicators 30. As illustrated in FIG. 2, the process monitoring system 10 may include a number N of readers 40 (e.g., 40₁, 40ₙ₋₁, 40ₙ). In some embodiments, the readers 40 may be coupled or stacked together and may have separate or integrated housings. Each reader 40 may include a different slot 42 for receiving a different cartridge 25 and associated process indicator 30.

The number N of readers 40 represents the bandwidth capacity of the process monitoring system 10 and may be selected, for example, to be sufficient to facilitate uninterrupted monitoring of a processing system 15 that is capable of performing multiple disinfection cycles in the time a reader 40 takes to read one process indicator 30. Including more than one reader 40 may be particularly useful in embodiments wherein each cartridge 25 includes a first process indicator 30 that is a chemical integrator and a second process indicator 31 that is a microorganism indicator 31 (e.g., biological indicator), and wherein each reader 40 is capable of reading both types of process indicators 30, 31. In some of these embodiments, each reader 40 may require more time to read a microorganism indicator 31 than the time required to complete a disinfection cycle in the processing system 15. The number N of readers 40 may be selected to facilitate uninterrupted verification of a selected number of disinfection cycles.

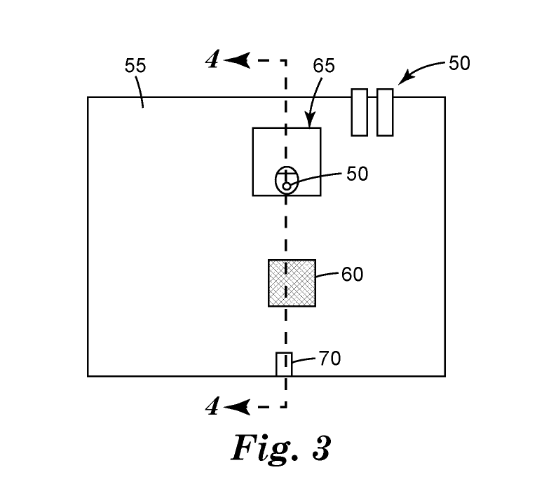

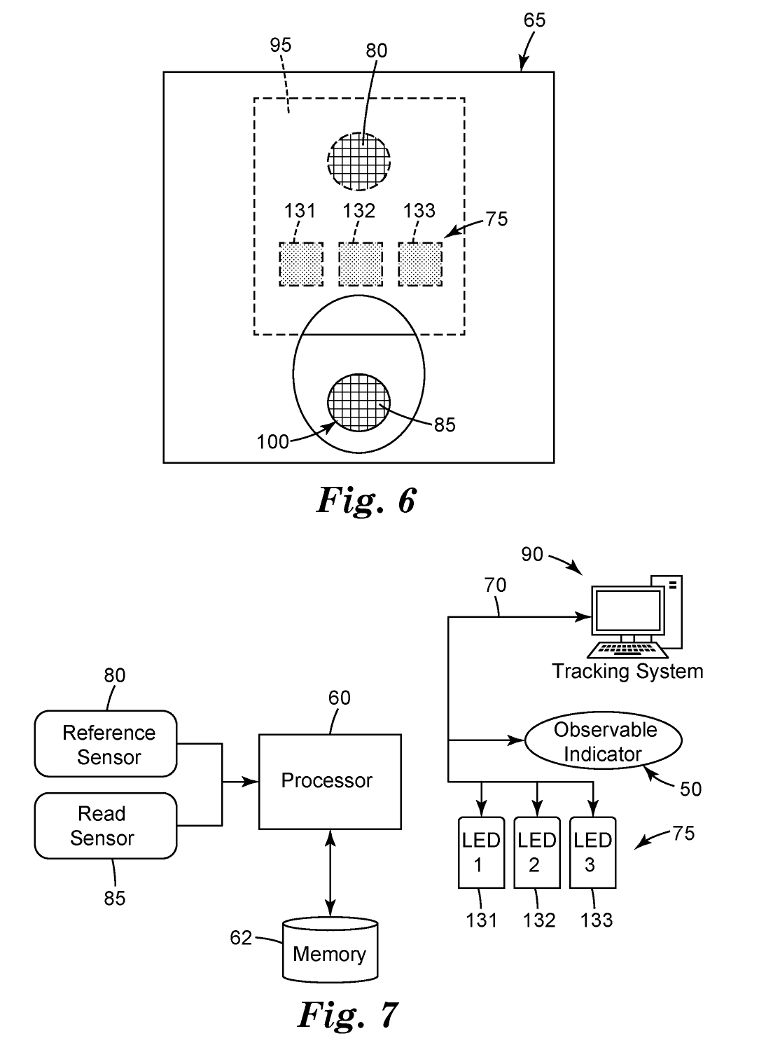

Further details of various elements and uses of the reader 40 are shown in FIG. 3 through FIG. 7. In the illustrated embodiment, the reader 40 can include a printed circuit board 55 coupled to the one or more observable indicators 50, a processor 60, memory 62, an optical block 65, a communication interface 70, a light source 75, a reference sensor 80, and a read sensor 85. The processor 60 can be operatively coupled in communication with the observable indicators 50, the memory 62, the communication interface 70, the light source 75, the reference sensor 80, and the read sensor 85.

The processor 60 can be configured to determine whether a process indicator 30 has a color representing an effective disinfection cycle. The processor 60 may provide a signal to the communication interface 70 representing whether the disinfection cycle was effective. A process tracking system 90 may also be operatively coupled to the communication interface 70. In response to the signal being provided to the communication interface 70, the process tracking system 90 can be configured to provide any of diagnostic assistance if the disinfection cycle was ineffective, updating of an inventory of available process indicators, and/or updating of a disinfection event database.

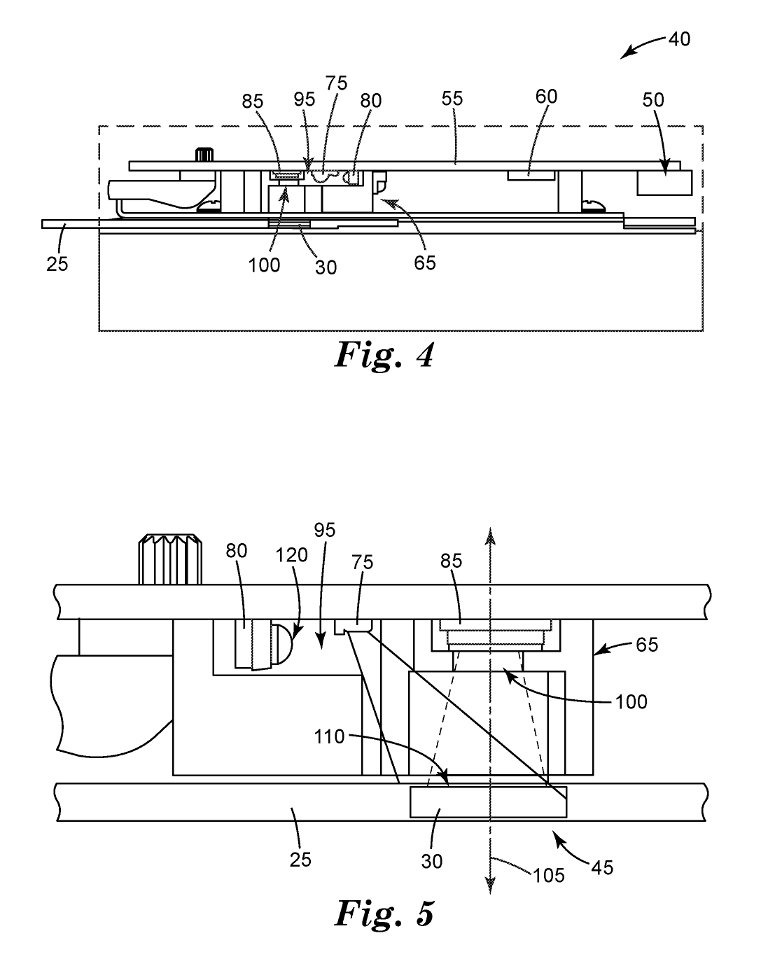

The process monitoring system 10 may further include an optical block 65 defining an interior volume 95 and an aperture 100, which are shown in FIG. 4 and FIG. 5. The aperture 100 may extend from the interior volume 95 to an exterior of the optical block 65. The light source 75, the reference sensor 80, and the read sensor 85 may be disposed in an interior volume of the optical block 65.

When in a reading position 45, the process indicator 30 can define an axis 105 normal to an indicator surface 110. As illustrated, the indicator surface 110 may be substantially planar.

The reader 40 may be designed to position the indicator surface 110 at a reading area of the reader 40 when the process indicator 30 is in the reading position 45. The reading area may be defined by the optical characteristics of the reader 40. The alignment of the light source 75, the read sensor 85, and the aperture 100 can contribute to defining the reading area.

When turned on, the light source 75 emits light to illuminate various surfaces in the reader 40. The reference sensor 80 may be positioned to receive light from the light source 75 that has not passed through the aperture 100. In some embodiments, the light source 75 emits light directly onto a surface 120 of the reference sensor 80. The reference sensor 80 may provide a reference sensor signal value that represents an intensity of light received at the reference sensor surface 120.

The light source 75 may illuminate the indicator surface 110 with light when the process indicator 30 is in the reading position 45. The light may reflect off of the indicator surface 110. Light emitted by light source 75 and reflected from the indicator surface 110 may be received by the read sensor 85 that has passed through the aperture 100.

The optical block 65 can be designed to prevent light emitted from the light source 75 from being directly received by the read sensor 85. There may be no line of sight between the read sensor 85 and the light source 75. The read sensor 85 may be positioned to receive light from the reading area. A surface 125 of the read sensor 85 can be directed toward the aperture 100 such that light emitted by the light source 75 reaching the read sensor surface 125 is reflected at least once. The read sensor 85 may also provide a read sensor signal value that represents an intensity of light received at the read sensor surface 125.

In various embodiments, the reader 40 may not include a beam splitter and/or a band pass filter. The light from the light source 75 may not pass through a beam splitter or a band pass filter before reaching the process indicator 30 or after being reflected from the process indicator 30 and reaching the read sensor surface 125.

The axis 105 extending through the indicator surface 110 can extend through the aperture 100 when the process indicator 30 is in the reading position. In some embodiments, the axis 105 may extend through the read sensor surface 125 of the read sensor 85 when the process indicator 30 is in the reading position 45. As illustrated, the read sensor 85 can be aligned with the aperture 100. In some embodiments, the axis 105 may not extend through the reference sensor surface 120 of the reference sensor 80 and/or does not extend through the light source 75 when the process indicator 30 is in the reading position 45.

The use of an off-axis position of the light source 75 and an on-axis position for the read sensor 85 may facilitate detection of diffuse light reflection rather than specular light reflection. This may be particularly advantageous when the cartridge 25 includes a laminate sheet covering the process indicator 30 (in order to form a fluid pathway) that would overwhelm the reflected light from the process indicator 30 with specular reflection from the laminate sheet. In some embodiments, a majority of reflected light reflected off the indicator surface 110 and received by the read sensor 85 is diffusely reflected light.

The light source 75 may have a viewing angle that is sufficiently wide to illuminate the indicator surface 110 and the reference sensor surface 120. The viewing angle may depend upon the exact placement of elements in the reader 40 or the shape of the optical block 65. In some embodiments, the light source 75 may emit light over at least a 60 degree viewing angle, at least a 90 degree viewing angle, at least a 120 degree viewing angle, or any other suitable viewing angle. In some embodiments, the light source 75 may include one or more light-emitting diodes (LEDs) each having a suitable viewing angle. In at least one embodiment, the LEDs are surface-mount LEDs.

In order to determine whether a disinfection cycle was effective, the light source 75 can be configured to illuminate the reading area with light at different wavelengths. In some embodiments, the light source 75 may be configured to illuminate at a first wavelength and a second wavelength. In at least one embodiment, the light source 75 may also be configured to illuminate at a third wavelength. Each wavelength may be provided by a different LED. In some embodiments, the light source 75 includes three LEDs, including a first LED 131, a second LED 132, and a third LED 133, each emitting light at a different wavelength. The processor 60 may provide signals to selectively turn on and/or turn off the LEDs of the light source 75 at the different wavelengths, independently.

When each LED is on, the other LEDs may be turned off. For each different wavelength, the processor 60 can receive a reference sensor signal value and a read sensor signal value generated from the reference sensor 80 and the read sensor 85, respectively. The sensor signal values may be indicative of the color of the process indicator 30. Whether the disinfection cycle was effective may be determined based at least in part on these sensor signal values.

The sensor signal values can indicate the color of the process indicator 30. The reference sensor 80 and the read sensor 85 may be uniform intensity sensors capable of providing a signal indicating the intensity of light across a spectrum bandwidth including the different wavelengths emitted by the light source 75. The process indicator 30 may be designed to absorb a greater amount of light at particular wavelengths if the disinfection cycle was effective. In other words, a percent reflectance of the process indicator 30 at one or more wavelengths may indicate whether the disinfection cycle was effective. Percent reflectance values may be calculated at one or more of the different wavelengths from the sensor signal values to facilitate a determination of the effectiveness of the disinfection cycle.

In some embodiments, the processor 60 can be capable of determining a first percent reflectance value for the first wavelength and a second percent reflectance value at the second wavelength. The processor 60 may also be capable of determining a third percent reflectance value for the third wavelength. The effectiveness determination may be based at least in part on one percent reflectance value achieving a threshold value. The effectiveness determination may also be based at least in part on two or more percent reflectance values, each achieving a different threshold value.

Percent reflectance values may be calculated, calibrated, and normalized from sensor signal values in various manners that further facilitate precise and accurate measurements of percent reflectance, as described herein in more detail.

In embodiments wherein the reader 40 is also capable of reading a microorganism indicator-type of process indicator 30, the reader 40 may further include a heater (e.g., incubator), an ultraviolet lightsource (e.g., UV LED), and a fluorescence detector (e.g., sensor with long pass filter). A reader 40 capable of reading a chemical indicator and a microorganism indicator may only require a user to place a cartridge 25 into one reader for both types of reads to advantageously verify disinfection with multiple techniques.

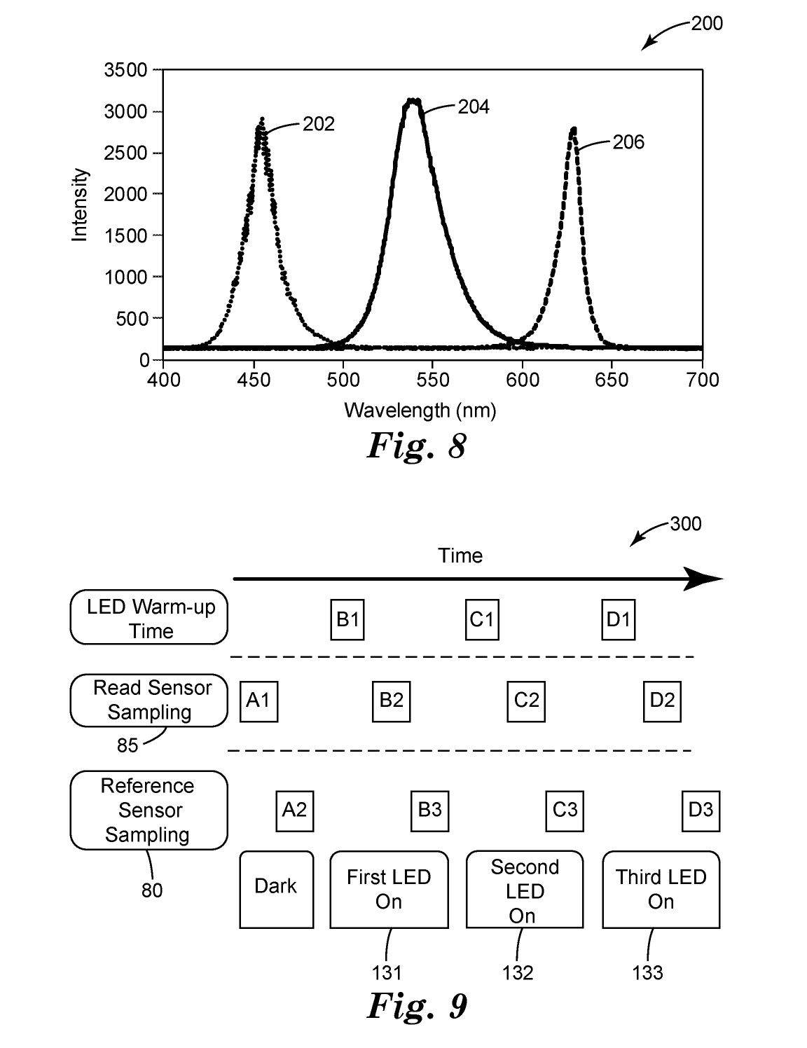

Examples of different wavelength intensities that can be emitted by the light source 75 are shown in the plot 200 of FIG. 8. As can be seen in the plot 200 of intensity distribution over wavelength (shown in nanometers), substantially non-overlapping wavelengths 202, 204, 206 can be provided for measurement by the sensors. Each wavelength 202, 204, 206 may be provided by a different LED 131, 132, 133. The wavelength of each LED can be identified as the peak value shown in the plot 200 of intensity distribution. As illustrated, the light source 75 may be configured to emit light at wavelengths of about 450 nm, about 530 nm, and about 630 nm. In some embodiments, a wavelength of about 660 nm may be used in lieu of 630 nm. However, any suitable wavelengths may be used depending on the type of process indicator 30 being used and the corresponding color change that represents a successful disinfection cycle.

The light source 75 may be selectively turned on and off in conjunction with measurements at the sensors, for example, by the processor 60. One example of a process 300 for performing a read by controlling the LEDs 131, 132, 133 and the sensors 80, 85 is shown in FIG. 9. In FIG. 9, dark measurements A1, A2 are taken from each of the read sensor 85 and the reference sensor 80, respectively. A dark measurement may be described as a measurement taken while all of the LEDs 131, 132, 133 of the light source 75 are turned off. The dark measurements A1, A2 generate values that can be received by the processor 60 and used to compensate for noise, or ambient light, from the percent reflectance value calculation. A dark reference sensor signal value and a dark read sensor signal value can be generated, for example, after the process indicator 30 is in the reading position 45 of the reader 40.

After the dark measurements A1, A2 are obtained, different LEDs 131, 132, 133 having different wavelengths are selectively turned on and off in sequence to generate corresponding reference sensor signal values and read sensor signal values that each represent the intensity of light received from the light source and reflected from the process indicator 30 in the reading position 45, respectively.

In measurements corresponding to a first wavelength, a first LED 131 is turned on while the other LEDs are off. In step B1, the first LED 131 is given time to warm-up (e.g., to reach steady state illumination). Then, in step B2, the read sensor 85 generates a read sensor signal value representing the intensity of reflected light from the reading area and the process indicator 30 emitted from the light source 75 a first wavelength. In step B3, the reference sensor 80 generates a reference sensor signal value representing the intensity of light from the light source 75 emitting the first wavelength. The processor 60 may only sample from one sensor in a time period.

Following the first wavelength measurements, measurements corresponding to the second wavelength C2, C3 may be taken with the second LED 132 turned on and emitting a second wavelength while the other LEDs are off. Then, corresponding measurements for the third wavelength D2, D3 may be taken with the third LED 133 turned on and emitting a third wavelength while the other LEDs are off.

The percent reflectance value for each wavelength may be calculated based on the measurements. Each percent reflectance value calculated can be proportional to the read sensor signal value divided by the reference sensor signal value (˜V_{read}/V_{ref}).

In some embodiments, the dark sensor signal values can be subtracted from the measured sensor signal values to compensate for offsets attributable, for example, to ambient light. The dark reference sensor signal value can be subtracted from each reference sensor signal value determined from steps B3, C3, D3 (V_{ref}−V_{darkref}). The dark read sensor signal value can also be subtracted from each read sensor signal value determined from steps B2, C2, D2 (V_{read}−V_{darkread}). The percent reflectance value calculated may then be proportional to the adjusted sensor signal values

$$( ∼ \frac{{V}_{read} - {V}_{darkread}}{{V}_{ref} - {V}_{darkref}} ) .$$

In some embodiments, the read sensor measurements A1, B2, C2, D2 and the reference sensor measurements A2, B3, C3, D3 represent sampling of the sensors, or multiple measurements. The multiple measurements can be used to further compensate for potential noise. In some embodiments, each sensor is sampled two or more (e.g., eight) times for each measurement. However, any number of suitable measurements can be performed to balance sampling time with accuracy or precision. The samples can be averaged, or otherwise combined, to generate a final sensor signal value used in the calculation of percent reflectance.

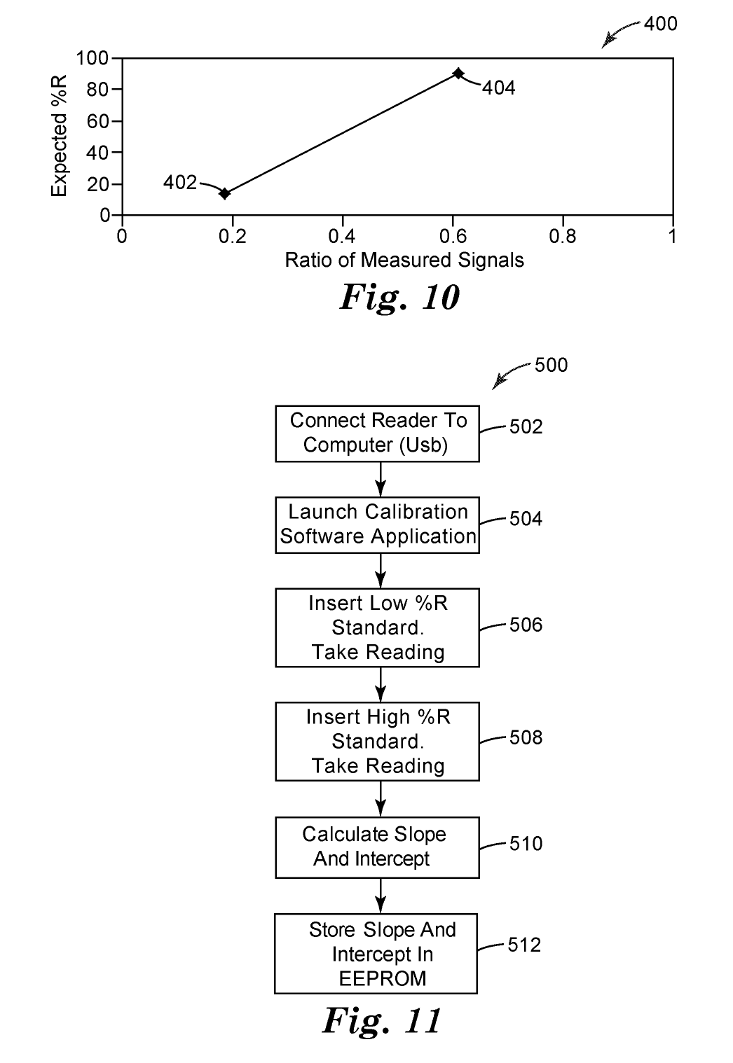

The reader 40 may also be calibrated, for example, before the measurements in process 300 are taken. One example correction function is represented by plot 400 of FIG. 10. A calibration may generate a correction function that may be stored in memory for later retrieval. One example process 500 for generating a correction function is shown in FIG. 11.

In some embodiments, a correction function compensates for differences between a measured percent reflectance value and an expected percent reflectance value. As shown, process 500 may begin in step 502 with connecting the reader 40 to a computer, for example, via a universal serial bus (USB) or any other suitable connection. In step 504, a calibration software may be launched on the computer that guides the user through the process of calibrating the reader 40 and communicates with the reader 40.

In steps 506 and 508, the software my prompt a user to position a calibration surface having a low percent reflectance value and a calibration surface having a high percent reflectance value in the reading position sequentially. Each calibration surface may include a commercially-available grayscale standard surface configured to provide a substantially uniform percent reflectance over a spectrum bandwidth (e.g., 400 to 700 nm). For example, for each wavelength in a spectrum bandwidth, a grayscale standard surface would reflect the substantially the same amount of light (e.g., the same % R independent of wavelength). Cartridges having calibration surfaces can be designed to be place grayscale standard surfaces into the reading position 45 of the reader 40 in the same manner as cartridge 25.

The predefined percent reflectance value of each calibration surface may be described as an expected percent reflectance value (expected % R). In other words, the calculated percent reflectance values based on the ratio of measured signals, such as

$$( \frac{{V}_{read} - {V}_{darkread}}{{V}_{ref} - {V}_{darkref}} ) ,$$

of a 3% R calibration surface is expected to correspond to 3% R. However, in many cases, the ratio of measured signal values may not be equal to the percent reflectance (e.g., 3% R may not correspond to a ratio of 0.3 of measured signals).

A low % R calibration surface and a high % R calibration surface can be used to provide two points in an expected % R versus a ratio of measured signals coordinate system. In step 506, a low % R calibration surface may be placed into the reading position 45 and measurements taken. As illustrated, a ratio of measured signals equals about 0.18 and corresponds to a calibration surface having about a 18% R expected value at point 402. In step 508, a high % R calibration surface may be placed into the reading position 45 and measurements taken. As illustrated, a ratio of measured signals equals about 0.62 and corresponds to a calibration surface having about a 95% R expected value at point 404.

From these two points, a slope and intercept can be calculated with a line fitting between points 402, 404 in step 510. A correction function based on the linear fitting preferably scales and shifts the ratio of measured signals so that accurate percent reflectance value can be generated in response to the measured signal values. One example of a correction function outputting a corrected percent reflectance value or calibrated percent reflectance value is shown in Equation 1:

$$Corrected \% R = ( \frac{{V}_{read} - {V}_{darkread}}{{V}_{ref} - {V}_{darkref}} ) \times slope - intercept$$

A correction function can be determined for each of the different wavelengths. In step 512, the one or more correction functions can be stored in a memory for later retrieval and use during a read of a process indicator 30.

After a read of a process indicator 30 and generation of a corrected percent reflectance value at a particular wavelength, the reader 40 may compare the corrected percent reflectance value to a threshold value (e.g., a particular % R value). If the corrected percent reflectance value meets the threshold, then the disinfection cycle may be described as having been effective. However, if the corrected percent reflectance value does not meet the threshold, then the disinfection cycle may be described as having been ineffective.

The particular wavelength selected may depend upon a process indicator type indicative of the measurements that can be used to determine whether the disinfection cycle was effective. Non-limiting examples of process indicator types include a processing system type, a disinfection cycle type, a chemical disinfectant type, a particular manufacturer of the process indicator and/or processing system, a calibration type, and combinations of two or more thereof. In some embodiments, the process indicator type may be represented by a type indicator on the cartridge 25. In some embodiments, the type indicator may be in the form of a physical feature, such as a predesigned hole/slot or the placement of a material having a different color than the cartridge body 27 in a specific location on the cartridge 25 (e.g., in a different location than the process indicator 30). In other embodiments, the type indicator may be in the form of a machine readable code, such as a barcode or QR code. In such embodiments, the process monitoring system 10 would be able to read the type indicator using a reading device that is separate from reading device 40.

In various embodiments, the reader 40 may measure more than one corrected percent reflectance value and determine whether a disinfection cycle was effective based at least in part on the more than one percent reflectance value. In one embodiment, the reader 40 can compare the corrected percent reflectance value at two or more wavelengths to corresponding thresholds. The two or more wavelengths may independently indicate the effectiveness of the disinfection cycle. In another embodiment, a % R versus wavelength slope may be determined and compared to a threshold slope (e.g., the slope may depend upon two or more % R measurements). In yet a further embodiment, a % R ratio may be determined and compared to a threshold ratio (e.g., a process indicator may have absorbed three times more at a first wavelength than at a second wavelength).

In some embodiments, the process indicator 30 is designed, before use, to be a white color, or to reflect at a substantially uniform and high level across the visible spectrum (e.g., 400 to 700 nm). However, process indicators 30 may absorb slightly differently at different wavelengths due to manufacturing variations, variations in distance of the process indicator 30 to the read sensor 85, and variations in the light source 75 output. To compensate for these potential variations, the cartridge 25 and reader 40 may be designed to facilitate robust alignment of the process indicator 30 with respect to the light source 75 and the read sensor 85, a constant current drive for the light source 75, and the use of dark measurements described herein. In addition, the corrected percent reflectance value at a first wavelength may be normalized against a corrected percent reflectance value at a second wavelength before being compared to a particular threshold value.

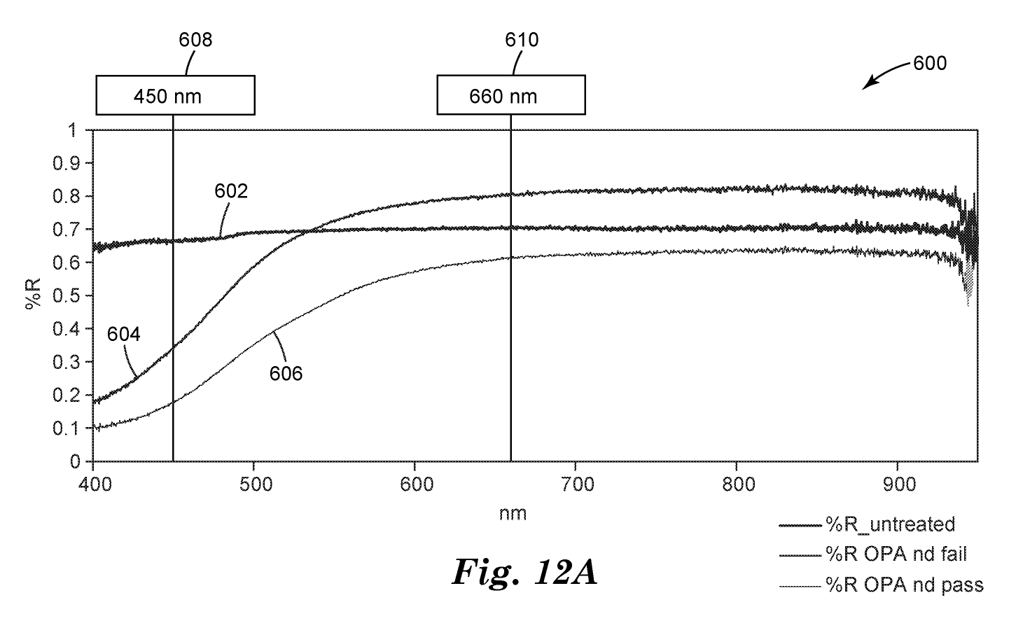

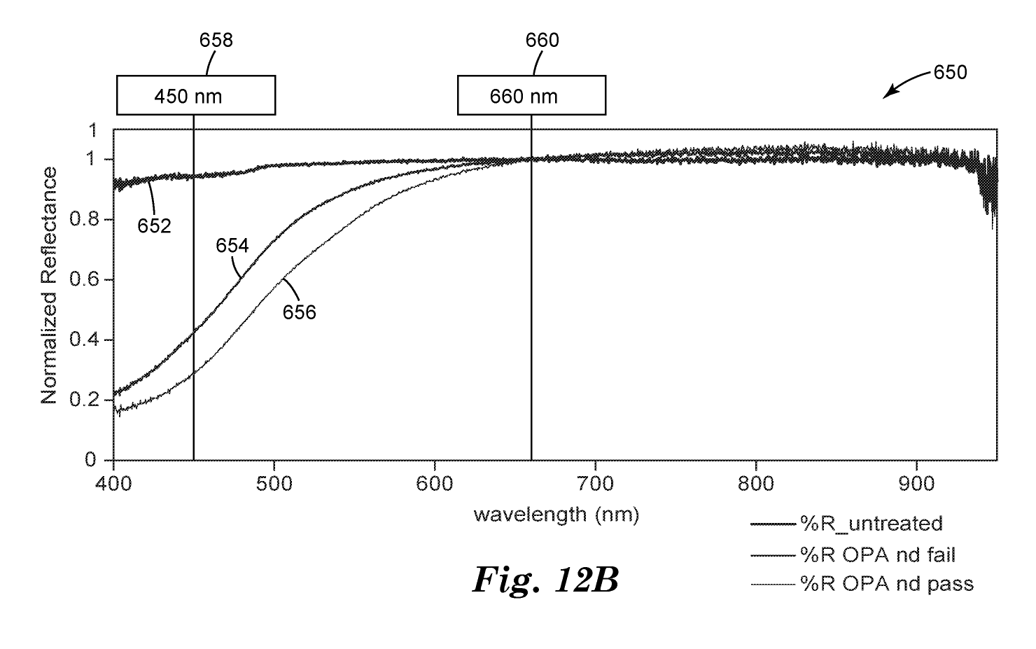

FIGS. 12A-B show the effect of normalizing the percent reflectance value at one wavelength based on another wavelength. Plot 600 shows measured % R values for an untreated process indicator 602 (e.g., a process indicator not subjected to a disinfection cycle), a fail process indicator 604 (e.g., a process indicator subjected to an ineffective disinfection cycle), and a pass process indicator 606 (e.g., a process indicator subjected to an effective disinfection cycle). As illustrated, a process indicator that represents an effective disinfection cycle 606 reflects less, or absorbs more, at one or more wavelengths versus the other process indicators 602, 604 that do not represent an effective disinfection cycle. However, there are differences in % R at a first wavelength 608 (440 nm) and a second wavelength 610 (660 nm) for each of the process indicators 602, 604, 606.

In some embodiments, the first % R at the first wavelength 608 may be normalized by the second % R second wavelength 610 by dividing the first % R by the second % R (% R1/% R2) at the respective wavelengths to remove any baseline offset as shown in plot 650. However, any suitable function may be used (e.g., subtraction, mapping to a different space, etc) to remove a baseline offset.

As illustrated, after the division process, the untreated process indicator 602 becomes normalized, untreated process indicator 652, the fail process indicator 604 becomes normalized fail process indicator 654, and the pass process indicator 606 becomes normalized pass process indicator 656. Although the entire visible spectrum is shown, the reader 40 may measure only at a first wavelength 608, 658 (450 nm) and a second wavelength 610, 660 (660 nm) based on the wavelengths emitted by the corresponding light source.

The first % R may be selected for normalization due to being more greatly affected by the disinfection cycle than the second % R (e.g., the % R difference between untreated and partially/fully treated process indicators is greater at 440 nm than at 660 nm). The second % R may represent a baseline % R that is expected to be relatively unaffected by the disinfection cycle. However, the second % R may also be normalized by the first % R. By normalizing the first % R with the second % R, change in % R can be more consistently determined. In some embodiments, a third % R at a third wavelength can also be measured and normalized by the second % R.

After the first % R is normalized, the normalized first % R may be compared against a threshold representing a normalized % R threshold. If the normalized first % R meets the threshold, then the disinfection cycle may be described as having been effective.

The normalized % R threshold may be predetermined and stored in a memory of the reader 40. The threshold may be determined, for example, by measuring the normalized % R at the first wavelength 608 for multiple pass process indicators and setting a threshold of 1, 2, or 3 standard deviations based on the sample. In another example, the threshold may be calculated based on the difference between a pass process indicator and a fail process indicator (e.g., set at the mean of the two values). In a further example, the threshold may be calculated based on the difference between a pass process indicator and an untreated process indicator (e.g., set at a percentage of the untreated % R value).

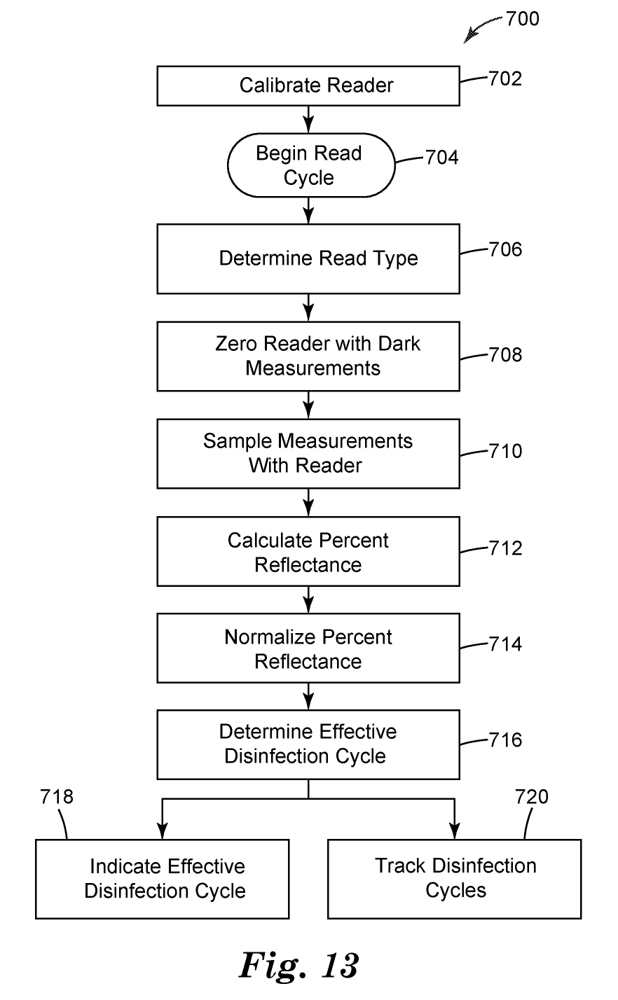

With the foregoing details being described, one example of a process 700 for using a reader is shown in FIG. 13. In step 702, a reader is calibrated and may store one or more correction functions in a memory. A user may then initiate the start of a read cycle 704, for example, by positioning a cartridge into a reading position of the reader. Calibration need not be performed before each read cycle (although it may be if so desired).

The reader may first determine a read type in step 706, for example, based on a process indicator type. The criteria for determining whether a disinfection cycle was effective may be based at least in part on the read type. In step 708, the reader is “zeroed” with dark measurements, which may be single or multiple samples. Dark measurement values may be stored in the reader and used to offset values measured while the light source is turned on.

In step 710, the reader performs a read by taking measurements with the light source turned on at one or more wavelengths. The measurements may be single or multiple samples. In step 712, a percent reflectance for each wavelength is calculated. An offset from the dark measurement values and a correction function based on the calibration may be applied to calculate each percent reflectance value.

In step 714, a percent reflectance is normalized. For example, a first percent reflectance may be scaled to a second percent reflectance. The normalized percent reflective may be compared to a threshold in step 716 to determine whether an effective disinfection cycle was performed by a processing system.

In step 718, an effective disinfection cycle may be indicated by the reader. The reader may also indicate that the disinfection cycle was ineffective. In step 720, the disinfection cycle may be tracked by a tracking system for purposes of diagnostics, inventory management, or compliance monitoring, for example.

In this manner, a process monitoring system 10 is described that can, in some embodiments, verify whether a process indicator represents a disinfection cycle that was effective within a few seconds or less (e.g., milliseconds), using low power consumption, at a low cost, and is relatively small.

The processors used in the process monitoring system described herein may be provided in any suitable form and may, for example, include a processing unit and optionally memory. In one or more embodiments, the processing unit of a processor may, for example, be in the form of one or more microprocessors, Field-Programmable Gate Arrays (FPGA), Digital Signal Processors (DSP), microcontrollers, Application Specific Integrated Circuit (ASIC) state machines, computing devices, etc. that may be integrated in a single piece of hardware or distributed in multiple pieces of hardware that can operatively communicate with one another.

The following embodiments are intended to be illustrative of the present disclosure and not limiting.

### Illustrative Embodiments

According to illustrative embodiment 1, a system includes: a reader configured to indicate whether an indicator surface of a process indicator has a color representative of an effective disinfection cycle when the process indicator is in a reading position of the reader, the process indicator defining an axis normal to the indicator surface, the reader having: an optical block defining an interior volume and an aperture, the axis extending through the aperture when the process indicator is in the reading position; a light source disposed in the interior volume and configured to illuminate the indicator surface with light, the axis not extending through the light source when the process indicator is in the reading position; optionally, a reference sensor positioned in the interior volume to receive, at a reference sensor surface, light from the light source that has not passed through the aperture, the reference sensor configured to provide a reference sensor signal value representing an intensity of light received, the axis not extending through the reference sensor surface when the process indicator is in the reading position; a read sensor disposed in the interior volume to receive, at a read sensor surface, light from the light source that has reflected off the indicator surface and has passed through the aperture, the read sensor configured to provide a read sensor signal value representing an intensity of reflected light received; and a processor operatively coupled to the light source, read sensor, and optional reference sensor, the processor configured to: receive the read sensor signal value and optionally the reference sensor signal value; and determine whether the process indicator has the color representative of an effective disinfection cycle based at least in part on the read sensor signal value and optionally the reference sensor signal value.

In illustrative embodiment 2, a system includes the system according to embodiment 1, wherein a majority of reflected light reflected off the indicator surface and received by the read sensor is diffusely reflected light.

In illustrative embodiment 3, a system includes the system according to any one of embodiments 1 to 2, wherein the light source has three light-emitting diodes, wherein each of the three light-emitting diodes is configured to emit light at a different wavelength.

In illustrative embodiment 4, a system includes the system according to any one of embodiments 1 to 3, wherein the light source is configured to emit light at wavelengths of about 450 nm, about 530 nm, and about 660 nm.

In illustrative embodiment 5, a system includes the system according to any one of embodiments 1 to 4, wherein the process indicator is configured to change color when subjected to one or more disinfection conditions, wherein the one or more disinfection conditions are selected from exposure time, temperature, disinfectant solution concentration, and any combination of two or more thereof.

In illustrative embodiment 6, a system includes the system according to any one of embodiments 1 to 5, wherein the process indicator is configured to change color when subjected to a disinfectant solution including a disinfectant selected from ortho-phthaldehyde, glutaraldehyde, peracetic acid, hypochlorous acid, hypochlorite, hydrogen peroxide, or any combination of two or more disinfectants thereof.

In illustrative embodiment 7, a system includes the system according to any one of embodiments 1 to 6, wherein the process indicator is a color change-based indicator.

In illustrative embodiment 8, a system includes the system according to any one of embodiments 1 to 7, wherein the light source includes a surface mount light-emitting diode having at least a 60 degree viewing angle.

In illustrative embodiment 9, a system includes the system according to any one of claims 1 to 8, and further includes a plurality of grayscale calibration standards, each grayscale calibration standard defining a different expected percent reflectance value, wherein the processor is further configured to store a percent reflectance correction parameter determined in response to measuring the percent reflectance value of each grayscale calibration standard at one or more of the different wavelengths.

In illustrative embodiment 10, a system includes the system according to any one of embodiments 1 to 9, wherein the light source emits light directly onto the reference sensor surface and wherein the read sensor surface is directed toward the aperture such that light emitted by the light source reaching the read sensor surface is reflected at least once.

In illustrative embodiment 11, a system includes the system according to any one of embodiments 1 to 10, wherein the axis extends through the read sensor surface when the process indicator is in the reading position.

In illustrative embodiment 12, a system includes the system according to any one of embodiments 1 to 11, and further includes a first observable indicator configured to inform a user that the disinfection cycle was effective.

In illustrative embodiment 13, a system includes the system according to any one of embodiments 1 to 12, and further includes a second observable indicator configured to inform a user that the disinfection cycle was ineffective.

In illustrative embodiment 14, a system includes the system according to any one of embodiments 1 to 13, and further includes a communication interface operatively coupled to the processor, wherein the processor is further configured to provide a signal to the communication interface representing whether the disinfection cycle was effective; and a process tracking system operatively coupled to the communication interface, wherein, in response to the signal being provided to the communication interface, the process tracking system is configured to provide at least one of the following: diagnostic assistance if the disinfection cycle was ineffective, updating of an inventory of available process indicators, and updating of a disinfection event database.

In illustrative embodiment 15, a method includes: generating a dark read sensor signal value from a read sensor and optionally a dark reference sensor signal value from an optional reference sensor after a process indicator is in a reading position of a reader; selectively turning on a light source of the reader at different wavelengths including a first wavelength and a second wavelength; generating, for each of the different wavelengths, a corresponding read sensor signal value and optionally a corresponding reference sensor signal value, each read sensor signal value representing an intensity of light reflected from the process indicator in the reading position and received by the read sensor, each reference sensor signal value representing an intensity of light received by the reference sensor from the light source; generating percent reflectance values, including a first percent reflectance value for the first wavelength and a second percent reflectance value for the second wavelength, each percent reflectance value being based at least in part on the corresponding read sensor signal value, the dark reference sensor signal value, the dark read sensor signal value, a corresponding predetermined correction function, and optionally the corresponding reference sensor value; normalizing the first percent reflectance value based at least in part on the second percent reflectance value; and determining whether the process indicator exhibits a color representative of an effective disinfection cycle based at least in part on the normalized first percent reflectance value and a corresponding predetermined threshold.

In illustrative embodiment 16, a method includes the method according to embodiment 15 and further includes: determining, for each of the different wavelengths, a correction function representing a relationship between a measured percent reflectance value and an expected percent reflectance value after a calibration standard is in the reading position of the reader; and storing each determined correction function for later retrieval.

In illustrative embodiment 17, a method includes the method according to any one of embodiments 15 to 16 and further includes: determining a process indicator type after the process indicator is in the reading position of the reader, wherein the process indicator type is selected from a reprocessing system type, a disinfection cycle type, a chemical disinfectant type, a particular manufacturer of the process indicator and/or processing system, a calibration type, and any combination of two or more thereof; and determining which of the different wavelengths to normalize in response to the process indicator type.

In illustrative embodiment 18, a method includes the method according to any one of embodiments 15 to 17 and further includes: positioning the process indicator in fluid communication with an adapter, the adapter being in fluid communication with a reprocessing machine and disinfectable equipment; running the disinfection cycle on the reprocessing machine after positioning the process indicator in fluid communication with the adapter; removing the process indicator from the adapter; and positioning the process indicator in the reading position of the reader.

In illustrative embodiment 19, an apparatus includes: a light source configured to illuminate a reading area with light at different wavelengths including a first wavelength and a second wavelength; a read sensor positioned to receive light from the reading area, the read sensor configured to provide a read sensor signal value representing an intensity of light received; optionally, a reference sensor positioned to receive light from the light source, the reference sensor configured to provide a reference sensor signal value representing an intensity of light received at the reference sensor surface; and a processor operatively coupled to the light source, read sensor, and optional reference sensor, the processor configured to: selectively turn on the light source at the different wavelengths; receive, for each of the different wavelengths, a read sensor signal value and optionally a reference sensor signal value; and determine whether a disinfection cycle was effective based at least in part on the read sensor signal values and optionally the reference sensor signal values.

In illustrative embodiment 20, an apparatus according to the apparatus of embodiment 19, wherein the processor is further configured to: determine a first percent reflectance value for the first wavelength and a second percent reflectance value at the second wavelength; and determine whether the disinfection cycle was effective based at least in part on a corresponding predetermined threshold for the first percent reflectance value, a corresponding predetermined threshold for the second percent reflectance value, or both.

In illustrative embodiment 21, an apparatus includes the apparatus according to embodiment 20, wherein the processor is further configured to: receive a dark reference sensor signal value and a dark read sensor signal value when the light source is off; and determine the percent reflectance values based at least in part on the dark reference sensor signal value and the dark read sensor signal value.

In illustrative embodiment 22, an apparatus includes the apparatus according to any one of embodiments 20 to 21, wherein the processor is further configured to: apply a corresponding correction function to each of the percent reflectance values; and determine whether the disinfection cycle was effective based at least in part on the corrected percent reflectance values.

In illustrative embodiment 23, an apparatus includes the apparatus according to any one of embodiments 20 to 22, wherein the processor is further configured to: determine, for each of the different wavelengths, a correction function representing a relationship between a measured percent reflectance value and an expected percent reflectance value after a calibration standard is positioned at the reading area; and store each determined correction function in a memory.

In illustrative embodiment 24, an apparatus includes the apparatus according to any one of embodiments 20 to 23, wherein the processor is further configured to: normalize the first percent reflectance value in response to the second percent reflectance value; and determine whether the disinfection cycle was effective based at least in part on the normalized first percent reflectance value.

Thus, embodiments of the READERS FOR PROCESS MONITORING SYSTEMS AND METHODS OF USE are disclosed. All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof. The disclosed embodiments are presented for purposes of illustration and not limitation.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the properties sought to be obtained by those skilled in the art utilizing the teachings disclosed herein.

The recitation of numerical ranges by endpoints includes all numbers subsumed within that range (e.g. 1 to 5 includes 1, 1.5, 2, 2.75, 3, 3.80, 4, and 5) and any range within that range. Herein, the terms “up to” or “no greater than” a number (e.g., up to 50) includes the number (e.g., 50), and the term “no less than” a number (e.g., no less than 5) includes the number (e.g., 5).

As used herein, “have”, “having”, “include”, “including”, “comprise”, “comprising” or the like are used in their open ended sense, and generally mean “including, but not limited to”. It will be understood that “consisting essentially of”, “consisting of”, and the like are subsumed in “comprising,” and the like.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

The term “and/or” means one or all of the listed elements or a combination of any two or more of the listed elements (e.g., casting and/or treating an alloy means casting, treating, or both casting and treating the alloy).

The phrases “at least one of,” “comprises at least one of,” and “one or more of” followed by a list refers to any one of the items in the list and any combination of two or more items in the list.

Reference to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout are not necessarily referring to the same embodiment of the disclosure. Furthermore, the particular features, configurations, compositions, or characteristics may be combined in any suitable manner in one or more embodiments.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The term “coupled” refers to two elements being attached to each other either directly (in direct contact with each other) or indirectly (having one or more elements between and attaching the two elements).

Terms related to orientation, such as “top”, “bottom”, “side”, and “end”, are used to describe relative positions of components and are not meant to limit the orientation of the embodiments contemplated. For example, an embodiment described as having a “top” and “bottom” also encompasses embodiments thereof rotated in various directions unless the content clearly dictates otherwise.

1. A system comprising:
a reader configured to indicate whether an indicator surface of a process indicator has a color representative of an effective disinfection cycle when the process indicator is in a reading position of the reader, the process indicator defining an axis normal to the indicator surface, the reader comprising:
an optical block defining an interior volume and an aperture, the axis extending through the aperture when the process indicator is in the reading position;
a light source disposed in the interior volume and configured to illuminate the indicator surface with light, the axis not extending through the light source when the process indicator is in the reading position;
optionally, a reference sensor positioned in the interior volume to receive, at a reference sensor surface, light from the light source that has not passed through the aperture, the reference sensor configured to provide a reference sensor signal value representing an intensity of light received, the axis not extending through the reference sensor surface when the process indicator is in the reading position;
a read sensor disposed in the interior volume to receive, at a read sensor surface, light from the light source that has reflected off the indicator surface and has passed through the aperture, the read sensor configured to provide a read sensor signal value representing an intensity of reflected light received; and
a processor operatively coupled to the light source, read sensor, and optional reference sensor, the processor configured to:
receive the read sensor signal value and optionally the reference sensor signal value; and
determine whether the process indicator has the color representative of an effective disinfection cycle based at least in part on the read sensor signal value and optionally the reference sensor signal value.

2. The system of claim 1, wherein a majority of reflected light reflected off the indicator surface and received by the read sensor is diffusely reflected light.

3. The system of claim 1, wherein the light source comprises three light-emitting diodes, wherein each of the three light-emitting diodes is configured to emit light at a different wavelength.

4. (canceled)

5. The system of claim 1, wherein the process indicator is configured to change color when subjected to one or more disinfection conditions, wherein the one or more disinfection conditions are selected from exposure time, temperature, disinfectant solution concentration, and any combination of two or more thereof.

6. (canceled)

7. The system of claim 1, wherein the process indicator is a color change-based indicator.

8. The system of claim 1, wherein the light source comprises a surface mount light-emitting diode having at least a 60 degree viewing angle.

9. The system of claim 1, further comprising a plurality of grayscale calibration standards, each grayscale calibration standard defining a different expected percent reflectance value, wherein the processor is further configured to store a percent reflectance correction parameter determined in response to measuring the percent reflectance value of each grayscale calibration standard at one or more of the different wavelengths.

10. The system of claim 1, wherein the light source emits light directly onto the reference sensor surface and wherein the read sensor surface is directed toward the aperture such that light emitted by the light source reaching the read sensor surface is reflected at least once.

11. The system of claim 1, wherein the axis extends through the read sensor surface when the process indicator is in the reading position.

12-13. (canceled)

14. The system of claim 1, further comprising:
a communication interface operatively coupled to the processor, wherein the processor is further configured to provide a signal to the communication interface representing whether the disinfection cycle was effective; and
a process tracking system operatively coupled to the communication interface, wherein, in response to the signal being provided to the communication interface, the process tracking system is configured to provide at least one of the following: diagnostic assistance if the disinfection cycle was ineffective, updating of an inventory of available process indicators, and updating of a disinfection event database.

15. A method comprising:
generating a dark read sensor signal value from a read sensor and optionally a dark reference sensor signal value from an optional reference sensor after a process indicator is in a reading position of a reader;
selectively turning on a light source of the reader at different wavelengths including a first wavelength and a second wavelength;
generating, for each of the different wavelengths, a corresponding read sensor signal value and optionally a corresponding reference sensor signal value, each read sensor signal value representing an intensity of light reflected from the process indicator in the reading position and received by the read sensor, each reference sensor signal value representing an intensity of light received by the reference sensor from the light source;
generating percent reflectance values, including a first percent reflectance value for the first wavelength and a second percent reflectance value for the second wavelength, each percent reflectance value being based at least in part on the corresponding read sensor signal value, the dark reference sensor signal value, the dark read sensor signal value, a corresponding predetermined correction function, and optionally the corresponding reference sensor value;
normalizing the first percent reflectance value based at least in part on the second percent reflectance value; and
determining whether the process indicator exhibits a color representative of an effective disinfection cycle based at least in part on the normalized first percent reflectance value and a corresponding predetermined threshold.

16. The method of claim 15, further comprising:
determining, for each of the different wavelengths, a correction function representing a relationship between a measured percent reflectance value and an expected percent reflectance value after a calibration standard is in the reading position of the reader; and
storing each determined correction function for later retrieval.

17. The method of claim 15, further comprising:
determining a process indicator type after the process indicator is in the reading position of the reader, wherein the process indicator type is selected from a reprocessing system type, a disinfection cycle type, a chemical disinfectant type, a particular manufacturer of the process indicator and/or processing system, a calibration type, and any combination of two or more thereof; and
determining which of the different wavelengths to normalize in response to the process indicator type.

18. The method of claim 15, further comprising:
positioning the process indicator in fluid communication with an adapter, the adapter being in fluid communication with a reprocessing machine and disinfectable equipment;
running the disinfection cycle on the reprocessing machine after positioning the process indicator in fluid communication with the adapter;
removing the process indicator from the adapter; and
positioning the process indicator in the reading position of the reader.

19. An apparatus comprising:
a light source configured to illuminate a reading area with light at different wavelengths including a first wavelength and a second wavelength;
a read sensor positioned to receive light from the reading area, the read sensor configured to provide a read sensor signal value representing an intensity of light received;
optionally, a reference sensor positioned to receive light from the light source, the reference sensor configured to provide a reference sensor signal value representing an intensity of light received at the reference sensor surface; and
a processor operatively coupled to the light source, read sensor, and optional reference sensor, the processor configured to:
selectively turn on the light source at the different wavelengths;
receive, for each of the different wavelengths, a read sensor signal value and optionally a reference sensor signal value; and
determine whether a disinfection cycle was effective based at least in part on the read sensor signal values and optionally the reference sensor signal values.

20. The apparatus of claim 19, wherein the processor is further configured to:
determine a first percent reflectance value for the first wavelength and a second percent reflectance value at the second wavelength; and
determine whether the disinfection cycle was effective based at least in part on a corresponding predetermined threshold for the first percent reflectance value, a corresponding predetermined threshold for the second percent reflectance value, or both.

21. The apparatus of claim 20, wherein the processor is further configured to:
receive a dark reference sensor signal value and a dark read sensor signal value when the light source is off; and
determine the percent reflectance values based at least in part on the dark reference sensor signal value and the dark read sensor signal value.

22. The apparatus of claim 20, wherein the processor is further configured to:
apply a corresponding correction function to each of the percent reflectance values; and
determine whether the disinfection cycle was effective based at least in part on the corrected percent reflectance values.

23. The apparatus of claim 20, wherein the processor is further configured to:
determine, for each of the different wavelengths, a correction function representing a relationship between a measured percent reflectance value and an expected percent reflectance value after a calibration standard is positioned at the reading area; and store each determined correction function in a memory.

24. The apparatus of claim 20, wherein the processor is further configured to:
normalize the first percent reflectance value in response to the second percent reflectance value; and determine whether the disinfection cycle was effective based at least in part on the normalized first percent reflectance value.
